(19) United States
(12) Patent Application Publication
(10) Pub. No.: US 2022/0078982 A1
(43) Pub. Date: Mar. 17, 2022

(54) DEVICE AND AGRICULTURAL DEVICE FOR MEASURING WATER PRESENT IN VEGETATION AND OPERATION METHOD THEREOF

(71) Applicant: INESC TEC - INSTITUTO DE ENGENHARIA DE SISTEMAS E COMPUTADORES, TECNOLOGIA E CIÊNCIA, Porto (PT)

(72) Inventors: Manuel Cândido DUARTE DOS SANTOS, Porto (PT); Filipe BATISTA NEVES DOS SANTOS, Porto (PT); Rodrigo GOMES BORGES, Porto (PT)

(21) Appl. No.: 17/420,060

(22) PCT Filed: Dec. 31, 2019
(86) PCT No.: PCT/IB2019/061469

§ 371 Date: Jun. 30, 2021

(30) Foreign Application Priority Data
Dec. 31, 2018 (PT) 115239
Mar. 28, 2019 (EP) 19166018.2

(51) Int. Cl. A01G 27/00; A01G 25/09; A01G 25/16; G01N 22/04; G01N 33/00

(52) U.S. Cl. CPC A01G 27/003; A01G 25/09; G01N 33/0098; G01N 22/04; A01G 25/16

(57) ABSTRACT

Device and agricultural device, and operation method thereof, for measuring plant water present in vegetation comprising: an microwave emitter of a displaceable beam; a microwave receiver; an electronic data processor connected to receive a signal from the microwave receiver; wherein the microwave emitter is arranged to direct said displaceable beam towards the microwave receiver for measuring attenuation of the beam passing through each of a plurality of locations of the vegetation; wherein the electronic data processor is configured for calculating the attenuation of the displaceable beam between the emitter and receiver to obtain a 2D image corresponding to the measurement of water present in the vegetation. In particular, said beam is displaceable by the device being mounted on a vehicle which is movable along the vegetation to be measured in a horizontal direction, the microwave emitter and microwave receiver being jointly mounted in opposite ends of an arched structure.

### TECHNICAL FIELD

The present disclosure relates to a device and agricultural device for measuring water present in vegetation using radio frequency attenuation, respective agricultural vehicle and operation method.

### BACKGROUND

Water plays a central role in farming processes and this input must be tightly controlled and corrective and precise actions must be taken to support healthy crops. Thus, precision agriculture together with agricultural robotics has been exploiting the use of variable rate technologies (VRT) for controlling the rate of agronomic inputs (e.g. water) based on contextual features such as plant status and location.

A traditional VRT system only uses information provided by agro data cloud systems to determine the amount of agronomic input required to be applied. However, these systems do not get real-time feedback about the real requirements of the plant needs (e.g. water) and the real amount of products applied.

Real-time and remote solutions (using satellites or aerial vehicles) disclosed in prior art comprise mostly radar scatterometers or optical-based systems.

Radar scatterometers namely Synthetic Aperture Radars (SAR), can monitor vegetation phenology, using an imaging-based system that transmits microwaves and receives the reflections of the backscattered signals. Although satellite SAR systems are specialized in providing average water content of wide planting regions they do not have resolution to measuring water content at a single plant or even at leave-by-leave-scale (i.e. precision agriculture applications). Moreover, satellite SAR systems are strongly affected by the interference of other elements during the scattering processes such as the soil moisture, the surface type, the terrain topology, the techniques used during the cultivation process and the atmospheric transmission characteristics [2].

Optical-based methods can be used for water sensing by analyzing the reflected electromagnetic signal by vegetation canopies. This reflectance is largely affected by the biological properties of the plant, and in the regions of 0.7 to 1.4 μm (near-infrared wavelengths) and 1.3 to 2.5 μm (middle-infrared wavelengths) of the electromagnetic spectrum, the reflectance is affected by the quantity of water in the leaves [1]. However, the accuracy of optical-based methods is negatively affected by atmospheric conditions. Moreover, water sensing using optical-based methods is limited to the contribution of the superficial layers of the plants because only reflected information is used.

Document KR101824652 relates to a device for calculating the moisture content of a plant. The device for calculating the weight and moisture content of a plant includes a cultivating bed on which a medium is mounted, wherein nutrient solution is supplied to enable the plant to be grown, a weight measuring portion, and a control portion. The weight measuring portion is configured to measure the weight of the medium mounted on the cultivating bed by floating the cultivating bed in the air. The control portion is configured to collect a measurement value from the weight measuring portion and calculate the weight and the water content of the plant by using the collected measurement value.

The prior art does not provide feasible practical solutions of measuring water content at a plant scale that can be adapted to different atmospheric and terrain conditions.

### REFERENCES

[1] E. R. Hunt and Barrett N Rock. Detection of changes in leaf water content using near- and middle-infrared reflectances. Remote Sensing of Environment, 30(1):43-54, 1989.
[2] G. Schiavon, D. Solimini, and A. Burini. Sensitivity of multi-temporal high resolution polarimetric C and L-band SAR to grapes in vineyards. In 2007 IEEE International Geoscience and Remote Sensing Symposium, pages 3651-3654, July 2007.

These facts are disclosed in order to illustrate the technical problem addressed by the present disclosure.

### GENERAL DESCRIPTION

This document discloses a method and device for measuring the water content in plants using radio frequency attenuation for real-time water sensing. The method comprises according to an embodiment transmitting electromagnetic radiation with frequencies in particular above 20 GHz on one side of a plant, receive the transmitted signal on the other side of the said plant, and analyze the attenuation of the signal caused by the water content of the plant being irradiated. The implemented approach includes analyzing the attenuation caused by leaves, plants, and elements containing water over a transmission between a pair of high gain antennas.

The presented method and device are immune to dry elements that obstruct the view or to illuminations changes, which would impair the measurement by an optical based system.

In an embodiment the system can be used in combination with a spraying system, analysing the plants right before each spraying passage and right after the said passage, giving a real-time accurate feedback of the amount of product sprayed.

In an embodiment of an anti-drift panel/drift recovery-based sprayer the system can monitor the water quantity right before the product application and monitor water quantity application on leaf and plant surfaces, without need to be in contact with the plants (as the optical based systems require).

The antenna array system can be attached to any place of mobile machinery (autonomous or non-autonomous) and/or in machinery implemented in such way that the plants will be in the middle of antenna array system. To avoid the collision between antenna array system and the plants an active pan and tilt system may be used to control the antenna array system posture.

It is disclosed a device for measuring plant water present in vegetation comprising:

a microwave emitter of a displaceable beam;
a microwave receiver;
an electronic data processor connected to receive a signal from the microwave receiver; wherein the microwave emitter is arranged to direct said displaceable beam towards the microwave receiver for measuring attenuation of the beam passing through each of a plurality of locations of the vegetation;
wherein the electronic data processor is configured for calculating the attenuation of the displaceable beam between the emitter and receiver to obtain a 2D image corresponding to the measurement of water present in the vegetation, that is, to obtain a spatial 2D image of the vegetation wherein the image corresponds to the calculated attenuation and hence the measurement of water present in the vegetation.

Alternatively, by adequate displacing of the beam (or beams) a 3D image can be obtained. Also, by steering or displacing the beam at an inclined angle, in relation to the horizontal plane, towards the vegetation, it is then obtained information in respect of the water measurement, along an horizontal axis along a direction towards the vegetation, in order to obtain a 3D image or a 2.5D image (a 3D image where there is limited information along one or more of 3 orthogonal axis).

In an embodiment, the microwave receiver comprises a plurality of individual receivers.

In an embodiment, the microwave receiver comprises a plurality of individual microwave receivers linearly arranged and distributed in particular along a vertical direction.

In an embodiment, the microwave emitter is arranged to direct said displaceable beam towards each of said individual microwave receivers.

In an embodiment, the microwave emitter is arranged to direct said displaceable beam towards one by one of said individual microwave receivers.

In an embodiment, the microwave emitter comprises a steerable beam antenna, in particular a steerable beam phase-shift antenna, for directing said microwave beam between each of said individual microwave receivers.

In an embodiment, the microwave emitter comprises a plurality of individual emitters which are individually switchable for directing said microwave beam between each of said individual microwave receivers.

In an embodiment, the microwave emitter comprises a plurality of individual microwave emitter linearly arranged and distributed in particular along a vertical direction.

In an embodiment, said beam is displaceable by the device being movable along the vegetation to be measured in particular along a horizontal direction, in particular the microwave emitter and microwave receiver being jointly mounted in the device, further in particular the microwave emitter and microwave receiver being jointly mounted in opposite ends of an arched structure such that the vegetation can pass below the arch between the emitter and the receiver.

In an embodiment, the device is a vehicle, namely an agricultural vehicle, and the microwave emitter and microwave receiver are mounted on the vehicle for moving along the vegetation.

An embodiment comprises a satellite geolocation sensor, a radiofrequency geolocation sensor or a displacement sensor for synchronizing the measurement of water present in the vegetation with the device location.

An embodiment comprises a second microwave emitter and a second microwave receiver mounted on the vehicle for moving along the vegetation, such that the second emitter-receiver pair measure attenuation of the beam passing through each of a plurality of locations of the vegetation after the first emitter-receiver pair have measured attenuation of the beam passing through each of the same plurality of locations of the vegetation.

In an embodiment, the electronic data processor is configured for calculating the difference in attenuation of the displaceable beam between the first emitter-receiver pair and the second emitter-receiver pair to obtain a 2D image corresponding to the difference in the measurement of water present in the vegetation.

An embodiment comprises a dispenser for applying the treatment and being arranged to:

measure the water present in the vegetation before applying the treatment and adjust the amount of treatment applied in respect of the measured water; and/or
measure the water present in the vegetation before and after applying the treatment for calculating the difference in the measurement of water present in the vegetation to measure the applied treatment.

It is also disclosed a method of operation of a device, namely an agricultural device, for measuring plant water present in vegetation, said device comprising a microwave emitter of a displaceable beam; a microwave receiver; an electronic data processor connected to receive a signal from the microwave receiver; said method comprising: directing said displaceable beam by the microwave emitter towards the microwave receiver for measuring attenuation of the beam passing through each of a plurality of locations of the vegetation;

calculating, by the electronic data processor, the attenuation of the displaceable beam between the emitter and receiver to obtain a 2D image corresponding to the measurement of water present in the vegetation.

### BRIEF DESCRIPTION OF THE DRAWINGS

The following figures provide preferred embodiments for illustrating the disclosure and should not be seen as limiting the scope of invention.

FIG. 1: Schematic representation of an overview of an embodiment of the disclosed device.

FIG. 2A and FIG. 2B: Schematic representations of views of possible embodiments using multiple emitting and receiving elements and their direct radiation pathways

FIG. 3: Schematic representation of a diagram of an embodiment of the antenna array with switches.

FIG. 4: Schematic representation of a view of an embodiment of antenna arrays with phase shifters.

FIG. 5: Schematic representation of a view of an embodiment of the disclosed device combined with a vehicle and a spraying system for agriculture applications.

FIG. 6: Comparison between the real image of the cactus-like plant and the resulting image after the analysis of the collected data obtained according to an embodiment of the disclosure.

FIG. 7: Leaf with a dried region (bottom) and the respective result of the test scan obtained according to an embodiment of the disclosure.

FIG. 8A, FIG. 8B, and FIG. 8C: Images of the analysis of a green leaf (A), the analysis of the same leaf but dry (B) and the difference between the two images (C) obtained according to an embodiment of the disclosure.

FIG. 9A, FIG. 9B, and FIG. 9C: Images of a green leaf before (A) and after having been sprayed with water hence with water on its surface (B) and the difference between the two images (C) obtained according to an embodiment of the disclosure.

### DETAILED DESCRIPTION

This document discloses a method and device for remote water sensing using transmitted microwaves, and more particularly for measuring water content in a plant. Unlike the optical-based methods that measure and analyse the visible and infrared signals reflected by the object of interest, the method and device of this document uses transmitted microwaves enabling sensing deeper plant layers and being immune to the interference caused by illumination changes or by the presence of dry elements on the accuracy of the predicted water content values.

The water content measurement can be used to characterize the water status of plants or as an input to the control other devices and systems (e.g. spraying system), adjusting automatically the amount of the agronomic inputs to be sprayed. The disclosed device and method is capable to create a 2D or 2.5D, or even three-dimensional, map of water content present in the plant or group of plants.

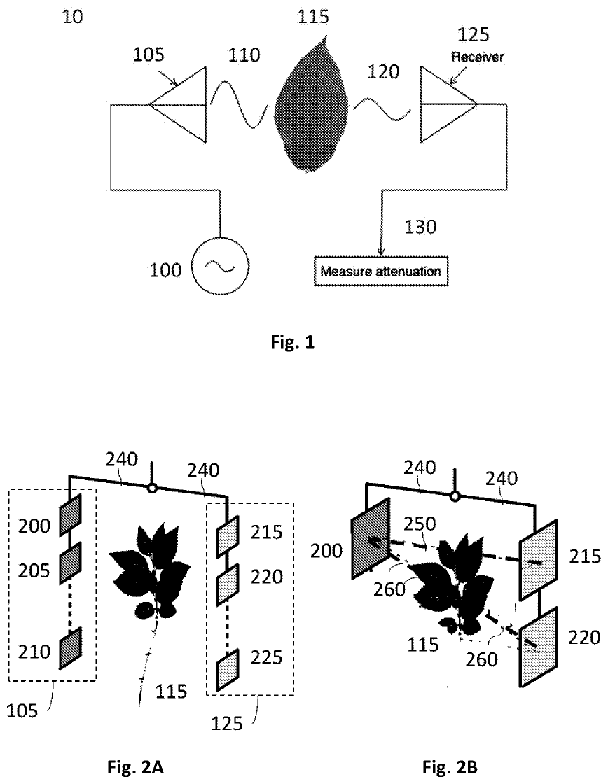

Reference is made to FIG. 1 which is a schematic view of the device and illustrates the method to measure water content in plants. As depicted by FIG. 1, the device 10 comprises an emitter 105 that generates an electromagnetic radiation 110 (e.g. microwaves) towards a plant or a region of the plant (e.g. leaves) 115.

The generated electromagnetic radiation 110 may be, but is not limited to, frequencies around 20 GHz, due to the effects of water presence in the signal radiation path, which is established between an emitter 105 and a receiver 125. In fact, the receiver measures a peak of attenuation around 20 GHz when water (gas or liquid form) or a water containing object is between the receiver and the emitter antennas (direct radiation path). The said electromagnetic radiation 110 is transmitted but attenuated along the plant 115. The transmitted electromagnetic radiation 120 is sensed by the receiver 125 and the signal attenuation is measured by the unit 130. The attenuation caused by the plant 115 leads to a loss in power of the said generated electromagnetic radiation 110, depending on the water content of the plant 115.

In free space conditions meaning absence of any object or plant 115 in the direct radiation path between the emitter and the receiver, the attenuation in the radiated signal is given by free space path loss (L_{fs}) calculated by the Equation 1, where f is the frequency, c is the speed of light in the vacuum and D is the distance between the emitter and the receiver.

$${L}_{fs} = 20 {log}_{10} ( \frac{4 π f D}{c} ) ( d B ) \qquad ( Equation 1 )$$

In presence of a water-containing object (e.g. plant) the measured signal loss between the emitter and the receiver is significantly increased when compared to the signal loss value obtained in the calibration, which can be performed in free space condition (LA or in a real environment (humid air) without a plant in the direct radiation path.

The plant water content is predicted by the differential amount of signal loss (attenuation) between the plant measure and the calibration.

Frequencies in the range of 20 GHz were applied in this example because they can go through dried plants without suffering signal loss so not changing L_{fs} value.

Higher frequencies than 20 GHz may be applied to improve water sensing sensitivity. However, higher frequencies lead to increased free space power loss what requires decreasing the distance between the emitter 105 and the receiver 125 for the same radiated power. Improved spatial resolution may be achieved by the use of higher frequencies than 20 GHz. Other different frequencies may be selected and used to avoid excessive losses due to specific use cases or to avoid reflections occurring at a specific frequency. Also, different frequencies may be used simultaneously, in order to electronically or computationally select the frequency signal or signals providing the best measurement.

In an alternative embodiment power reflection may be also used to predict the plant water content. However, the power reflected by the said plant is extremely low which leads to decreased sensitivity comparing to the disclosed method using attenuation between the emitter and the receiver.

In order to water sensing different plants with different shapes and sizes, a variable number of emitters and receivers can be used as disclosed in FIG. 2A. In this embodiment, the emitter element 105 may be an array comprising at least an emitter antenna 200 and the receiver element 125 may be an array with at least a receiver antenna 215. The term emitter/receiver pair will be used to describe any combination of an emitter from a multitude of emitters in an array and a receiver from a multitude of receivers in an array that can establish a link.

The said emitter array 105 and the said receiver array 125 are connected by a structure 240 (e.g. metallic bar) to control the distance between them, i.e. the direct radiation path length. The said structure 240 may be connected to a mobile structure (e.g. robotic arm) that can move the said emitter and receiver arrays to measure the water content in different points of the plant 115.

Reference is made to FIG. 2B to explain the different radiation paths that characterize each emitter/receiver pair. For example, the radiation path 250 characterize the link between the emitter 200 and the receiver 215 while the radiation path 260 characterize the link between the emitter 200 and the receiver 220. Therefore, the same emitter can provide different radiation paths which can be sensed by different receivers. This increase in the number of direct radiation paths can be used to improve the spatial resolution and the accuracy of the measurement because each direct radiation path between an emitter/receiver pair can be used to measure an attenuation value, and thus water content, that can be mapped. This map can be presented as a water content image.

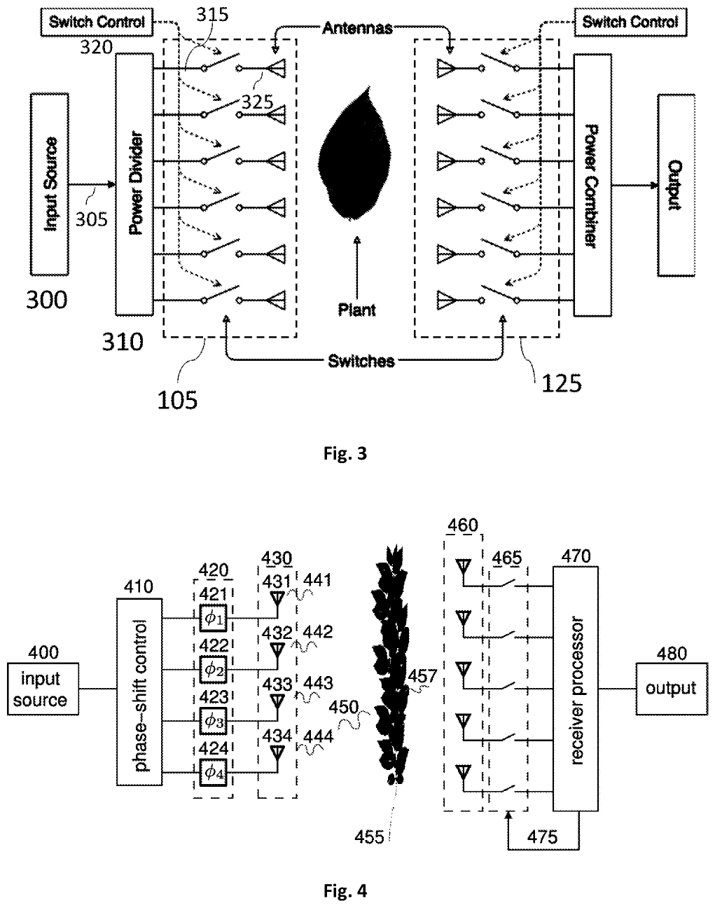

To mitigate the interference caused by reflections on the water sensing, this disclosure implements isolation between signals paths by controlling along time the operation of non-simultaneous emitter/receiver pairs. Reference is made to FIG. 3 to explain how the emitter/receiver pairs can be established.

References is made to FIG. 3 to disclose the electric-scan based in switch controlling. An input source 300 generates the input signal 305 which is injected in a power divider 310. This power divider 310 is composed of as many power channels 315 as the number of emitting antennas. Each power channel 315 is connected to a switch control 320 that controls the power delivered to each emitting antenna 325.

Each emitter/receiver pair is activated at each given moment, with the intensity of the emitted signal being attenuated by the objects and plants in the signal path between the emitter/receiver pair. In this embodiment, the electrical scan consists of controlling the switches connected to each antenna as shown in FIG. 3 turning on and off the different emitter/receiver pairs in the arrays to scan the whole area.

Reference is made to FIG. 4 to describe a preferable embodiment of the disclosure disclosed herein comprising electric-scan based in phase shifting for beam steering control. An input source 400 generates the input signal which is injected in a phase-shift control 410. This phase-shift control 410 is composed of as many power channels as the number of emitting antennas. Each power channel supplies a phase shifter 421-424. A phase control module 410 individually controls each phase shifter 421-424. Each phase shifter 421-424 is connected to an emitting antenna 431-434 that emits an emitted signal 441-444. At least two phase shifters 421 and 422 and correspondent two emitting antennas 431 and 432 are required for steering the emitted signals 441-442 in to an emitted beam 450.

This electronically steerable array of antennas enables the control of the direction of the emitted beam 450 (beam). In this configuration, all the emitting antennas of the array 450 emit at the same time. The combination of the individual emitted signals of each antenna of the array creates an emitted beam, directing the radiation pattern of the array in a specific direction.

The emitted beam angle control is achieved by shifting the phase of the signal emitted from each emitting antenna to provide constructive or destructive interference so as to steer the emitted beam 450 in the desired direction. The direction of the emitted beam 450 is therefore changed by controlling the phase shifters 420 connected to each radiating elements of the array 430.

The beam steering of the array disclosed in the FIG. 4 is performed by controlling the phase of the signal for each antenna. The direction of the beam is defined by the phase difference between each consecutive element of the array. This phase difference (Δφ) is obtained using the Equation 2, where d is the predetermined distance between the radiating elements, λ is the wavelength (e.g. 20 GHz) and θₛ is the desired angle of the beam.

$$Δφ = \frac{360 ° \times d \times sin ( {θ}_{s} )}{λ} \qquad ( Equation 2 )$$

The emitted beam 450 suffers attenuation while it is transmitted along the plant 455, continuing as transmitted beam 457 towards the receiving elements 460. Each receiving element is connected to a switch 465, which is controlled by switch control 475, turning on and off individual switches from 465. The receiver processor 470 determines the water content value, based in measured the attenuation values, to each element of the map. At each time the receiver processor 470 has access to the present emitted beam angle which can be used to weight or correct the map of attenuations and consequent water content values that are outputted 480.

The determined attenuation (power loss) values are normalized to the range of 0 to 1 using the maximum and minimum values of the matrix with the absolute values. A grayscale image of the object, plant or leaf is therefore generated where the brightest part of the image represents the area with the greatest attenuation and, therefore, a greater water quantity, whereas, the darkest areas of the image represents a lower signal attenuation corresponding to areas with less water content.

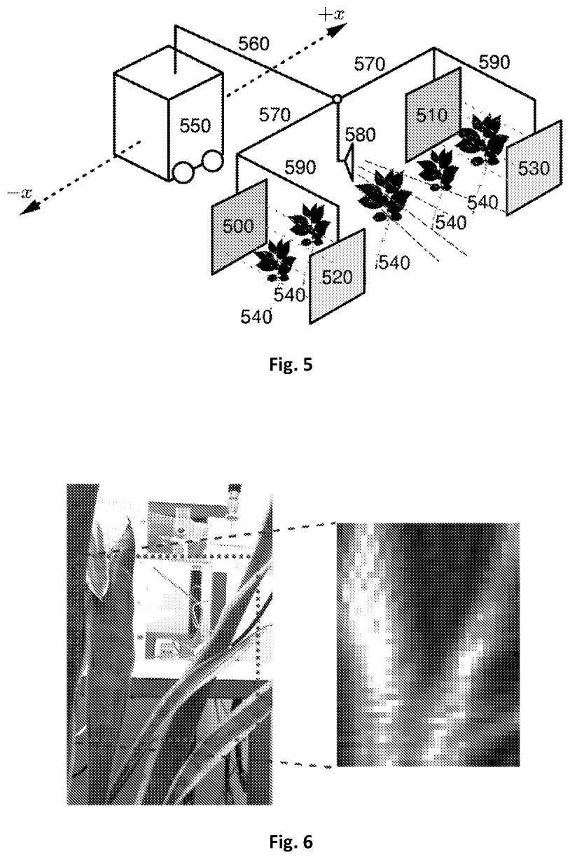

Reference is made to FIG. 5 to disclose a preferential embodiment of the present disclosure for agriculture applications. In this embodiment a vehicle 550 is connected through an element 560 to an apparatus to automatically spray 580 an agronomical product. In this embodiment, and assuming that the said vehicle 550 is moving along the positive x axis, the plant 540 water content is determined by the device that comprises the emitter/receiver pair 510 and 530, connected to a fixed structure 590 to ensure a constant distance, prior to the spray step. A second emitter/receiver pair 500 and 520 is used to provide a second water content measurement that is carried out after the spraying step. The elements 500 and 520 are arranged in face-to-face manner where the plant 540 is between them. The emitter/receiver pair are attached to the sprayer element 580 through an element 570. The plant water content value determined by the pair 510 and 530 can be used to control the amount of agronomical product sprayed by the element 580. The vegetation elements 540 are sprayed using a sprayer 580 that is attached to a vehicle 550 that moves in parallel with the alignment of the vegetation (x axis).

Based on the differences of attenuation (water content) sensed by the two said emitter/receiver pairs (510, 530 and 500, 520) the amount of sprayed agronomical product is determined as well as its position and scattering on the plant considering the data collected during the scanning step (beam steering). In case of low product distribution, the vehicle 550 moves backwards, repeating the spraying step. Each transmitter transmits a signal with transmit power and known frequencies. The positioning of the vehicle is known over time. Along the +/−x offset each receiver that records the attenuation over time. According to the direction of travel of the vehicle 550, one receiver will record the power of the received signal before spraying and the other the power of the signal after spraying. The difference in power attenuation received, after correction of distance between receivers, allows to determine the quality of spraying.

The longitudinal velocity of the vehicle can be changed in order to increase or decrease the longitudinal map detail as data collection is synchronised with the displacement of the vehicle, for example by satellite or radiofrequency geolocation, or for example by a rotational encoder placed at the vehicle wheels or transmission.

The signal attenuation that has a direct relation to the water quantity is integrated with the system geolocation to build a three-dimensional map where each observed spot, defined in a North-East-Up referential coordinate system. This three-dimensional signal attenuation map is after converted to a three-dimensional water content map.

In an alternative embodiment, the present disclosure can be used to continuous monitoring of plant water content as it is possible to repeat previous scans to obtain data over time.

Typically, measurements can be obtained at multiple times per second, which enables sampling rates of for example 1000 measurements per metre of vehicle displacement, such that the horizontal (in the direction of displacement) sampling points per metre can be larger or even much larger than the vertical (along the distribution of antenna emitter/receiver pairs) sampling points per metre. As a consequence, the horizontal image resolution can be larger or much larger than the vertical image resolution. Advantageously, this data can be used by computer methods to produce more detailed data on plant growth.

In an embodiment, fruit content can also be measured as fruits contain water. In a further embodiment, fruit water content can be also discriminated from leaf water content as fruits will cause much larger signal attenuation. In another further embodiment, fruit water content can be also discriminated from leaf water content as a vehicle applies a phytosanitary treatment by taking two consecutive measurements and using the fact that leaves will cause differential measurements reflecting the water content of the treatment applied whilst fruit will cause the signal to reach a maximum signal threshold and thus no differential measurement.

The following pertains to experimental results. The laboratorial tests carried out to evaluate the method and device disclosed herein used two high gain antennas mounted on a claw. This claw was placed on the end of a robotic arm, which allowed for the scanning of the object being measured. The pair of high gain antennas was connected to a vector network analyzer (VNA) by coaxial cables to measure the S-parameters of the transmitted signal, more precisely, the S₁₂ parameter. The model of the VNA was the E8363B PNA Series Network Analyzer (10 MHz to 40 GHz) from Agilent Technologies and the model of the coaxial cables were the KMM24 from Thorlabs and the KBL-2FT-PHS+ from Minicircuits, both prepared for the high frequencies in use during the tests.

For each test, a test element was placed between the emitter/receiver pair to be scanned. The area of this scanning process was defined in the Matlab script with the desired step size. The maximum value of this step size was 3 mm, dictated by the maximum precision of the robotic arm. The maximum size of the scanning area was a 20 cm by 20 cm square, due to physical limitations of the robotic arm. The test sequence consisted of acquiring the S₁₂ parameter measured by the VNA on the initial point, send a request to move the robotic arm to the next position, acquire the S₁₂ parameter of that position and so on until all the positions defined by the pre-determined area for the scanning process were achieved.

Water content measurements were carried out in different objects, plants, and leaves. The size of the objects and plants was limited by the distance between the emitter/receiver pair. This distance was dictated by the size of the two claws used. With the first claw, the distance between antennas was 20 cm, whereas, with the second claw, this distance was 9 cm. Initially, the tests were performed using the first claw, although the distance between the antennas meant that sometimes the VNA measured really low values, in the range of −70 dB to −80 dB for the S₁₂ parameter. This value was at the end of the VNA measuring range, and for that reason, the second claw was created to improve the tests performance and to increase the range of intensity values acquired by the VNA.

The different tests performed with plants and leaves will be presented in the following paragraphs. The leaves used during the tests were suspended from a wooden structure so that the only object in the middle of the emitter/receiver pair was the leaves. The same type of leaf was used throughout the tests to decrease the number of variables. As for the plant, its size was small enough to be fitted between the antennas and to allow for the scanning procedure.

A test using cactus-like plant was performed, as presented in FIG. 6. This plant was chosen due to its high content in water, causing a greater attenuation on the intensity of the signal. The analysis of the collected data for this test confirms that the signal is strongly attenuated when the signal is passing through the plant. This is perceptible by analyzing the images obtained from the collected data, where the brightest parts of the images correspond to areas where a leaf of the plant is directly in the middle of the emitter/receiver pair, and the darkest areas correspond to the void spaces between each leaf, as observed in FIG. 6. The scanning area of this test was a rectangle of 20 cm by 15 cm with a step size of 5 mm, corresponding to 1200 different points. The results are from three different frequencies (19.5 GHz, 19.75 GHz and 20 GHz) as well as the average of the attenuation in the range of 19.5 to 20 GHz. The analysis of the resulting images reveals better results using the average of the frequency range.

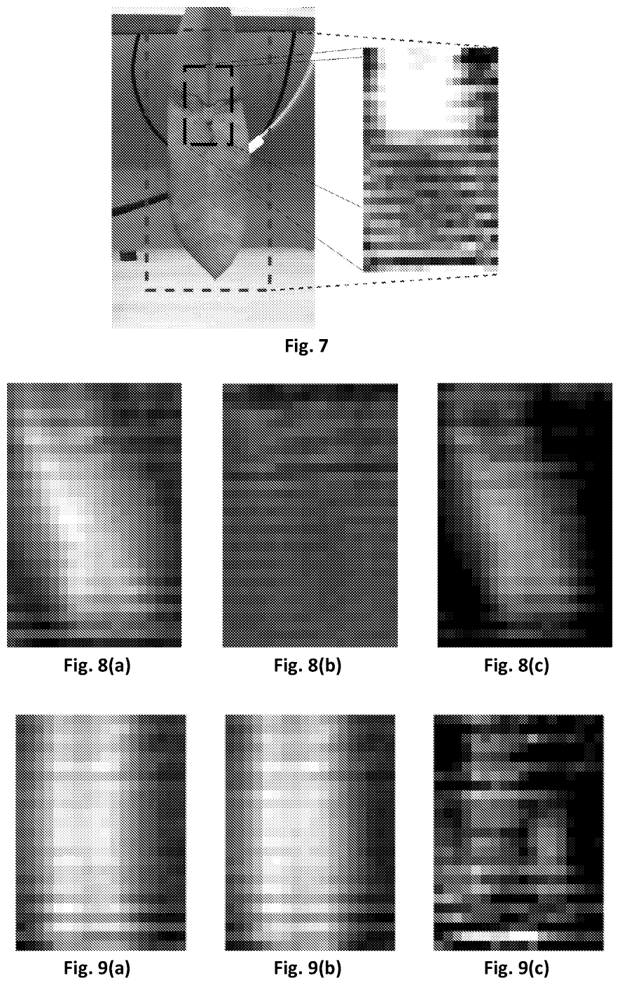

The method and device disclosed herein is able to scan and measure in detail the water content of object of interest with regions with different hydration levels. The disclosure was tested using a leaf with a green region (upper, leaf petiole) and a dry region (bottom, leaf apex) as illustrated on the left side of FIG. 7. The leaf was directly extracted from the tree in this condition, with almost half of the leaf in a dry state. The scanning area of this test was a rectangle of 12 cm by 7 cm with a step size of 0.4 cm, corresponding to 540 different points. As observed in FIG. 7, the disclosed invention measured higher water content and further presented brighter pixels in the upper region of the generated 2D image, which corresponds to the green region observed near to the leaf petiole. Conversely, the middle and bottom part of the generated image comprised darker pixels, which resulted from the reduced signal attenuation suffered by the microwaves transmitted along this dry region of the leaf near the apex that was measured by the disclosure. This correlates to the real image since the brown portion of the leaf has less water content in comparison to the green part.

The disclosure is also able to measure water content along time. This test consisted of starting with a green leaf freshly taken from a tree, measure its mass using a high precision scale and perform a scanning test. Next, the leaf was left to dry naturally for two days, before being again weighted and scanned. For this test, the size of the scanning area was a 12 cm by 8 cm rectangle with a step size of 0.4 cm, corresponding to a total of 600 different points.

The weight of the leaf decreased between each scanning procedure, starting at 1.1094 grams and finishing at 0.3908 grams. This loss of weight by the leaf is mainly due to the evaporation of water during the natural drying process, since the physical structure of the leaf stayed intact during the drying process and the scanning procedures. This loss of weight corresponds to a loss of water by the leaf, thus affecting each test scan results. The results of this analysis are shown in FIG. 8, where FIG. 8a represents the result of the scan of the green leaf (with a mass of 1.1094 grams), FIG. 8b represents the result of the dry leaf (with a mass of 0.3908 grams) and image FIG. 8c is the difference between the results of the green leaf and the dry leaf. The images were obtained by normalizing both matrices with the global maximum and minimum between the matrices acquired from both scanning procedures. The fact that the green leaf result has a brighter area in the zone of the leaf, whereas, on the dry leaf the result is an image almost black and uniform, indicates a clear sensibility of the system to the water content inside the leaf. The lack of a clear contrast between the dry leaf and the area of the image without the leaf in FIG. 8b indicates that there is not a significant attenuation of the signal due to the lack of water on the dry leaf.

The analysis of the sum of all the elements of the normalized matrices also gives a clear indication of the effects of the water content of the leaf on the signals' attenuation. The sum of the values of the normalized matrix of the green leaf was 262.20, whereas, for the dry leaf this sum was 156.47. This difference is explained due to the fact that a greater attenuation of the signal is represented by a number closer to 1 on the matrix, thus giving a bigger sum of the elements of the matrix of the green leaf.

The analysis of the difference between the maximum and minimum values recorded by the VNA also reveals some information about the leaves water content. For the data recorded for the green leaf, the difference between the maximum and the minimum values was 6.59 dB, whereas, on the dry leaf, this value was 2.57 dB. This difference of 4.02 dB between these two values reveals a clear indication of a greater effect on the signals' attenuation with the green leaf.

The same test was repeated using a smaller distance between antennas, this time with the scanning procedure being repeated six times over the course of two days and the mass of the leaf being measured before each scan. The values of the leaves mass for each scanning procedure are shown in Table 1, with the first three scans being performed on the first day and the following scans on the second day.

TABLE 1
Leaves mass at each scan (test) and corresponding
difference between the maximum and minimum values
of the matrices of each test result.

| | Day 1 | | | Day 2 | | |
|---|---|---|---|---|---|---|
| | Test 1 | Test 2 | Test 3 | Test 4 | Test 5 | Test 6 |
| Mass (g) | 1.0472 | 0.9741 | 0.9336 | 0.6181 | 0.5652 | 0.5311 |
| Difference | 3.87 | 3.74 | 3.73 | 1.92 | 1.86 | 1.58 |
| Max − Min | | | | | | |
| (dB) | | | | | | |

The analysis of the difference between the maximum and minimum values of the data collected for each test reveals an indication of the effect of the leaves water content on the signal attenuation. The higher the value of the leaves mass (corresponding to a higher water content), the higher the attenuation on the signals' intensity, represented by a greater difference between the maximum and minimum values of the of the tests performed on the first day in relation to the tests on the second day, as shown in Table 1.

Reference is made to FIG. 9 to disclose the results on water sensing after applying a water-based agricultural treatment. The purpose of this test was to check the sensitivity of the disclosure to small changes in the quantity and position of water on a green leaf. The scanning area of this test was a 10 cm by 7 cm rectangle with a step size of 0.4 cm, corresponding to 450 different points. Water content was measured at two different timepoints—the first immediately after water spraying (FIG. 9a) and the second, two hours and half later (FIG. 9b). Despite the small visual difference between FIG. 9a and FIG. 9b, the resulting difference of these two images shows some bright pixels in the leaf apex that is shown in the lower part of the image (FIG. 9c). These pixels correspond to a leaf region where the signal attenuation due to the presence of water in the leaf is higher. These bright pixels are located in the leaf apex (lower part of FIG. 9c) as expected since gravity pulls down the drops of water towards the apex. The fact that the test took about two and a half hours to be completed was also a factor to this result as some of the water on the top part of the leaf evaporated before the scan passed over that part of the leaf. The analysis of the pixel values of each image for the two timepoints also revealed that there is a bigger sum for the matrix of the leaf with water on its surface, which again confirms the effect of water on the signals' attenuation. The sum of the values of this matrix was 242.453, whereas, for the matrix of the values for the leaf without water, this sum corresponds to 230.723.

Preferably, for avoiding parallax measurement errors, the beam is unfocused, propagating in constant width, i.e. constant spatial beam profile—from each emitter, through the respective vegetation location to be measured, to the respective receiver (i.e. without concentration or spreading of the beam).

When using a vehicle, the horizontal resolution may be variable, for example taking into account the precision of the geolocation or the displacement sensor. Thus, the horizontal and vertical resolution may differ when using a vehicle.

The term “comprising” whenever used in this document is intended to indicate the presence of stated features, integers, steps, components, but not to preclude the presence or addition of one or more other features, integers, steps, components or groups thereof.

It is to be appreciated that certain embodiments of the disclosure as described herein may be incorporated as code (e.g., a software algorithm or program) residing in firmware and/or on computer useable medium having control logic for enabling execution on a computer system having a computer processor, such as any of the servers described herein. Such a computer system typically includes memory storage configured to provide output from execution of the code which configures a processor in accordance with the execution. The code can be arranged as firmware or software to configure the machine in which it is executed to perform the associated functions, as described herein.

The disclosure should not be seen in any way restricted to the embodiments described and a person with ordinary skill in the art will foresee many possibilities to modifications thereof. The above described embodiments are combinable. The following claims further set out particular embodiments of the disclosure.

1. A device for measuring plant water present in vegetation, comprising:
a microwave emitter of a beam, arranged in that the beam is displaceable to pass through each of a plurality of locations of the vegetation;
a microwave receiver; and
an electronic data processor connected to receive a signal from the microwave receiver and to control the displacement of said beam,
wherein the microwave emitter is arranged to direct said displaceable beam towards the microwave receiver for measuring attenuation of the beam passing through each of said plurality of locations of the vegetation, and
wherein the electronic data processor is configured to calculate the attenuation of the displaceable beam between the emitter and receiver and to obtain from the calculation a spatial 2D image of the vegetation wherein the image corresponds to the measurement of water present in the vegetation.

2. The device for measuring plant water present in vegetation according to claim 1, wherein the microwave receiver comprises a plurality of individual receivers.

3. The device for measuring plant water present in vegetation according to claim 1, wherein the microwave receiver comprises a plurality of individual microwave receivers linearly arranged and distributed, such that each receiver receives the beam passing through each of said plurality of locations of the vegetation.

4. The device for measuring plant water present in vegetation according to claim 2, wherein the microwave emitter is arranged to direct said displaceable beam towards each of said individual microwave receivers.

5. The device for measuring plant water present in vegetation according to claim 2, wherein the microwave emitter is arranged to direct said displaceable beam one by one toward said individual microwave receivers, such that each receiver receives the beam passing through each of said plurality of locations of the vegetation.

6. The device for measuring plant water present in vegetation according to claim 2, wherein the microwave emitter comprises a steerable beam antenna configured to direct said microwave beam between each of said individual microwave receivers.

7. The device for measuring plant water present in vegetation according to claim 2, wherein the microwave emitter comprises a plurality of individual emitters which are individually switchable for directing said microwave beam between each of said individual microwave receivers.

8. The device for measuring plant water present in vegetation according to claim 1, wherein the microwave emitter comprises a plurality of individual microwave emitters linearly arranged and distributed such that each receiver receives the beam emitted from each of said emitters passing through each of said plurality of locations of the vegetation.

9. The device for measuring plant water present in vegetation according to claim 1, wherein said beam is displaceable by the device being movable along the vegetation to be measured along a horizontal direction, the microwave emitter and microwave receiver being jointly mounted in the device.

10. An agricultural vehicle comprising the device for measuring plant water present in vegetation according to claim 1, wherein the microwave emitter and microwave receiver are mounted on the vehicle for moving along the vegetation such that the beam is displaceable to pass through each of the plurality of locations of the vegetation as the vehicle moves.

11. The agricultural vehicle according to claim 10, further comprising a satellite geolocation sensor, a radiofrequency geolocation sensor or a displacement sensor for synchronizing the measurement of water present in the vegetation with the device location determining which the plurality of locations of the vegetation is being measured as the vehicle moves.

12. The agricultural vehicle according to claim 10, wherein the microwave emitter and microwave receiver mounted on the vehicle define a first pair, the vehicle further comprising a second microwave emitter and a second microwave receiver mounted on the vehicle for moving along the vegetation, such that the second emitter-receiver defines a second pair that measures attenuation of the beam passing through each of a plurality of locations of the vegetation after the first emitter-receiver pair have measured attenuation of the beam passing through each of the same plurality of locations of the vegetation.

13. The agricultural vehicle according to claim 12, wherein the electronic data processor is configured for calculating a difference in attenuation of the displaceable beam between the first emitter-receiver pair and the second emitter-receiver pair to obtain a 2D image corresponding to the difference in the measurement of water present in the vegetation.

14. The agricultural vehicle according to claim 10, further comprising a dispenser for applying a water-based agricultural treatment to vegetation, the vehicle being arranged to:
measure the water present in the vegetation before applying the treatment and to adjust the amount of treatment applied in respect of the measured water; or
measure the water present in the vegetation before and after applying the treatment to calculate the difference in the measurement of water present in the vegetation to measure the applied treatment or
to both (1) measure the water present in the vegetation before applying the treatment and to adjust the amount of treatment applied in respect of the measured water and measure the water present in the vegetation after applying the treatment to calculate the difference in the measurement of water present in the vegetation to measure the applied treatment.

15. A method for operating an agricultural device that measures plant water present in vegetation, said device comprising a microwave emitter of a beam arranged in that the beam is displaceable to pass through each of a plurality of locations of the vegetation; a microwave receiver; and an electronic data processor connected to receive a signal from the microwave receiver and to control the displacement of said beam; said method comprising:
moving said displaceable beam by the microwave emitter towards the microwave receiver for measuring attenuation of the beam passing through each of a plurality of locations of the vegetation;
calculating, by the electronic data processor, the attenuation of the displaceable beam between the emitter and receiver; and
obtaining from the calculating step a spatial 2D image of the vegetation, wherein the image corresponds to the measurement of water present in the vegetation.
